(19) United States
(12) Patent Application Publication
(10) Pub. No.: US 2009/0053150 A1
(43) Pub. Date: Feb. 26, 2009

(54) Aerosol device containing at least one amps polymer and at least one fixing polymer

(76) Inventors: Charles Gringore, Paris (FR); Francoise Pataut, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/662,143

(22) PCT Filed: Sep. 8, 2005
(86) PCT No.: PCT/FR05/02337

§ 371 Date: Sep. 11, 2007

Related U.S. Application Data
(60) Provisional application No. 60/620,424, filed on Oct. 21, 2004.

(30) Foreign Application Priority Data
Sep. 9, 2004 (FR) 0452005

(51) Int. Cl. A61K 8/04; A61K 8/81; A61Q 5/06

(52) U.S. Cl. 424/47

(57) ABSTRACT

The invention concerns a composition packaged in an aerosol container, containing (c) at least a propellant and (b) a hair-styling composition including at least (i) one non polyurethane anionic or nonionic fixing polymer and (ii) at least one polymer comprising at least one ethylenically unsaturated monomer having a sulphonic group, in free form, or partly or totally neutralized.

The present invention relates to an aerosol device comprising at least one polymer of the AMPS type and at least one fixing polymer and to a styling method using such a device.

Styling compositions packaged in the form of an aerosol spray generally comprise a large alcohol fraction. In point of fact, cosmetic products with a high alcohol content are coming under particular scrutiny, in particular in the United States, as a result of public opinion recently becoming sensitive to the ecological problems resulting from the emission of volatile organic products to the atmosphere.

One solution which makes it possible to reduce the amount of alcohol, indeed even to completely eliminate alcohol from the formulations, is the replacement by an equivalent amount of water. However, this introduction of large amounts of water into aerosol sprays intended for the fixing of the hair, such as lacquers, results in a significant undesirable change in the shape of the hair and in a deterioration in the cosmetic properties. Furthermore, the majority of propellants of hydrocarbon type are incompatible with water and their use in compositions with a high water content is for this reason generally impossible.

Styling products are generally used to introduce a lasting hold to the hairstyle.

For this, fixing polymers are introduced in an aqueous/alcoholic medium.

Packaging in the form of an aerosol makes possible uniform distribution over the whole of the hair.

The fixing of the hairstyle is then virtually immediate and it is not easy, indeed even impossible, to structure the hairstyle with the hands as the hairs are instantaneously set rigid.

The problem posed by the invention is that of supplying a hair cosmetic composition which suitably fixes the hair, which provides good cosmetic properties and which dries in a reasonable time, while being distributed well and while contributing a feeling of freshness on application.

A subject matter of the present invention is a composition packaged in an aerosol device, comprising

(a) at least one propellant and
(b) a styling composition comprising at least (i) one nonpolyurethane anionic or nonionic fixing polymer and (ii) at least one polymer comprising at least one ethylenically unsaturated monomer comprising a sulfo group in the free or partially or completely neutralized form.

Another subject matter of the invention is a styling method which consists in vaporizing the mixture present in said aerosol device, either directly over the hair to be shaped or via the fingers, and in allowing the hair thus treated to dry.

### Styling Composition

All anionic and nonionic fixing polymers and their mixtures used in the art can be used in the compositions according to the present patent application.

The term “fixing polymer” is understood, within the meaning of the present invention, as meaning any polymer capable of contributing a form retention of the hair in a given form.

### Fixing Polymers

### Anionic Fixing Polymers

The fixing polymers may be soluble in the cosmetically acceptable medium or may be insoluble in this same medium and, in this case, used in the form of dispersions of solid or liquid polymer particles (latex or pseudolatex).

The anionic fixing polymers generally used are polymers comprising groups derived from carboxylic, sulfonic or phosphoric acid and have a number-average molecular mass of between approximately 500 and 5 000 000.

The carboxyl groups are contributed by unsaturated mono- or dicarboxylic acid monomers such as those corresponding to the formula:

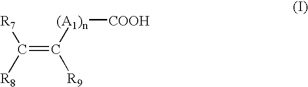

in which n is an integer from 0 to 10, A₁ denotes a methylene group, optionally connected to the carbon atom of the unsaturated group or to the neighboring methylene group when n is greater than 1 via a heteroatom, such as oxygen or sulfur, R₇ denotes a hydrogen atom or a phenyl or benzyl group, R₈ denotes a hydrogen atom or a lower alkyl or carboxyl group, and R₉ denotes a hydrogen atom, a lower alkyl group or a —CH₂—COOH, phenyl or benzyl group.

In the abovementioned formula, a lower alkyl group preferably denotes a group having 1 to 4 carbon atoms and in particular methyl and ethyl groups.

The preferred anionic fixing polymers comprising carboxyl groups according to the invention are:

A) Homo- or copolymers of acrylic or methacrylic acid or their salts and in particular the products sold under the names Versicol® E or K by Allied Colloid and Ultrahold® by BASF, the copolymers of acrylic acid and of acrylamide sold in the form of their sodium salts under the names Reten 421, 423 or 425 by Hercules or the sodium salts of polyhydroxycarboxylic acids.
B) Copolymers of acrylic or methacrylic acid with a monoethylenic monomer, such as ethylene, styrene, vinyl esters or esters of acrylic or methacrylic acid, optionally grafted onto a polyalkylene glycol, such as polyethylene glycol, and optionally crosslinked. Such polymers are disclosed in particular in French patent No. 1 222 944 and German application No. 2 330 956, the copolymers of this type comprising, in their chain, an optionally N-alkylated and/or -hydroxyalkylated acrylamide unit, such as disclosed in particular in Luxembourgian patent applications Nos. 75370 and 75371 or provided under the name Quadramer by American Cyanamid. Mention may also be made of copolymers of acrylic acid and of C₃-C₄ alkyl methacrylate and terpolymers of vinylpyrrolidone, of acrylic acid and of C₁-C₂₀ alkyl methacrylate, for example lauryl methacrylate, such as that sold by ISP under the name Acrylidone® LM, and methacrylic acid/ethyl acrylate/tert-butyl acrylate terpolymers, such as the product sold under the name Luvimer® 100 P by BASF.

Mention may also be made of the methacrylic acid/acrylic acid/ethyl acrylate/methyl methacrylate copolymers in aqueous dispersion sold under the name Amerhold® DR 25 by Amerchol.

C) Copolymers of crotonic acid, such as those comprising, in their chain, vinyl acetate or propionate units and optionally other monomers, such as allyl or methallyl esters, vinyl ether or vinyl ester of a linear or branched saturated carboxylic acid comprising a long hydrocarbon chain, such as those comprising at least 5 carbon atoms, it optionally being possible for these polymers to be grafted or crosslinked, or alternatively another monomer which is a vinyl, allyl or methallyl ester of an α- or β-cyclic carboxylic acid. Such polymers are disclosed, inter alia, in French patents Nos. 1 222 944, 1 580 545, 2 265 782, 2 265 781, 1 564 110 and 2 439 798. Commercial products coming within this class are the Resins 28-29-30, 26-13-14 and 28-13-10 sold by National Starch.
D) Copolymers of C₄-C₈ monounsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, or acrylic acid and its esters, the anhydride functional groups of these copolymers optionally being monoesterified or monoamidated. Such polymers are disclosed in particular in U.S. Pat. Nos. 2,047,398, 2,723,248 and 2,102,113 and GB patent No. 839 805. Commercial products are in particular those sold under the names Gantrez® AN or ES by ISP;
copolymers comprising (i) one or more maleic, citraconic or itaconic anhydride units and (ii) one or more monomers chosen from allyl or methallyl esters, optionally comprising one or more acrylamide, methacrylamide, α-olefin, acrylic or methacrylic ester, acrylic or methacrylic acid, or vinylpyrrolidone groups in their chain, the anhydride functional groups of these copolymers optionally being monoesterified or monoamidated.

These polymers are, for example, disclosed in French patents Nos. 2 350 384 and 2 357 241 of the applicant company.

E) Polyacrylamides comprising carboxylate groups.

The homopolymers and copolymers comprising sulfo groups are polymers comprising vinylsulfonic, styrenesulfonic, naphthalenesulfonic or acrylamidoalkylsulfonic units.

These polymers can in particular be chosen from;
salts of polyvinylsulfonic acid having a molecular weight of between approximately 1000 and 300 000, as well as copolymers with an unsaturated comonomer, such as acrylic or methacrylic acids and their esters, as well as acrylamide or its derivatives, vinyl ethers and vinylpyrrolidone;
salts of polystyrenesulfonic acid, such as the sodium salts sold, for example, under the name Flexan® 500 and Flexan® 130 by National Starch. These compounds are disclosed in patent FR 2 198 719;
mentioned in U.S. Pat. No. 4,128,631 and more particularly the polyacrylamidoethylpropanesulfonic acid sold under the name Cosmedia Polymer HSP 1180 by Henkel.

Mention may be made, as other anionic fixing polymer which can be used according to the invention, of the anionic branched block polymer sold under the name Fixate G-100 by Noveon.

According to the invention, the anionic fixing polymers are preferably chosen from acrylic acid copolymers, such as the acrylic acid/ethyl acrylate/N-tert-butylacrylamide terpolymers sold in particular under the name Ultrahold® Strong by BASE, copolymers derived from crotonic acid, such as the vinyl acetate/vinyl tert-butylbenzoate/crotonic acid terpolymers and the crotonic acid/vinyl acetate/vinyl neododecanoate terpolymers sold in particular under the name Resin 28-29-30 by National Starch, polymers derived from maleic, tumaric or itaconic acids or anhydrides with vinyl esters, vinyl ethers, vinyl halides, phenylvinyl derivatives, or acrylic acid and its esters, such as the monoesterified methyl vinyl ether/maleic anhydride copolymers sold, for example, under the name Gantrez® by ISP, the copolymers of methacrylic acid and of methyl methacrylate sold under the name Eudragit® L by Rohm Pharma, the copolymers of methacrylic acid and of ethyl acrylate sold under the name Luvimer® MAEX or MAE by BASF, the vinyl acetate/crotonic acid copolymers sold under the name Luviset CA 66 by BASF, the vinyl acetate/crotonic acid copolymers grafted by polyethylene glycol sold under the name Aristoflex® A by BASF or the polymer sold under the name Fixate G-100 by Noveon.

Preference is more particularly given, among the anionic fixing polymers mentioned above, to the use, in the context of the present invention, of the monoesterified methyl vinyl ether/maleic anhydride copolymers sold under the name Gantrez® ES 425 by ISP, the acrylic acid/ethyl acrylate/N-tert-butylacrylamide terpolymers sold under the name Ultrahold® Strong by BASF, the copolymers of methacrylic acid and of methyl methacrylate sold under the name Eudragit® L by Rohm Pharma, the vinyl acetate/vinyl tert-butyl-benzoate/crotonic acid terpolymers and the crotonic acid/vinyl acetate/vinyl neododecanoate terpolymers sold under the name Resin 28-29-30 by National Starch, the copolymers of methacrylic acid and of ethyl acrylate sold under the name Luvimer® MAEX or MAE by BASS, the vinylpyrrolidone/acrylic acid/lauryl methacrylate terpolymers sold under the name Acrylidone® LM by ISP or the polymer sold under the name Fixate G-100 by Noveon.

### Nonionic Fixing Polymers

The nonionic fixing polymers which can be used according to the present invention are chosen, for example, from;
polyalkyloxazolines;
vinyl acetate homopolymers;
vinyl acetate copolymers, such as, for example, copolymers of vinyl acetate and of acrylic ester, copolymers of vinyl acetate and of ethylene, or copolymers of vinyl acetate and of maleic ester, for example of dibutyl maleate;
acrylic ester homopolymers and copolymers, such as, for example, copolymers of alkyl acrylates and of alkyl methacrylates, such as the products provided by Rohm & Haas under the names Primal® AC-261 K and Eudragit® NE 30 D, by BASS under the name 8845 or by Hoechst under the name Appretan® N9212;
copolymers of acrylonitrile and of a nonionic monomer chosen, for example, from butadiene and alkyl (meth)acrylates; mention may be made of the products provided under the name CJ 0601 B by Rohm & Haas;
styrene homopolymers;
styrene copolymers, such as, for example, copolymers of styrene and of alkyl(meth)acrylate, such as the products Mowilith® LDM 6911, Mowilith® DM 611 and Mowilith® LDM 6070 provided by Hoechst or the products Rhodopas® SD 215 and Rhodopas® DS 910 provided by Rhône-Poulenc, copolymers of styrene, of alkyl methacrylate and of alkyl acrylate, copolymers of styrene and of butadiene or copolymers of styrene, of butadiene and of vinylpyridine;
polyamides;
vinyllactam homopolymers other than vinylpyrrolidone homopolymers, such as the polyvinylcaprolactam sold under the name Luviskol® Plus by BASF; and
vinyllactam copolymers, such as the poly(vinylpyrrolidone/vinyllactam) copolymer sold under the trade name Luvitec® VPC 55K65W by BASF, poly(vinyl-pyrrolidone/vinyl acetate) copolymers, such as those sold under the name PVPVA® 5630L by ISP or Luviskol® VA 73, VA 64, VA 55, VA 37 and VA 28 by BASF, and poly(vinylpyrrolidone/vinyl acetate/vinyl propionate) terpolymers, such as, for example, that sold under the name Luviskol® VPA 343 by BASF.

The alkyl groups of the nonionic polymers mentioned above preferably have from 1 to 6 carbon atoms.

According to the invention, it is also possible to use fixing polymers of grafted silicone type comprising a polysiloxane part and a part composed of a silicone-free organic chain, one of the two parts constituting the main chain of the polymer and the other being grafted onto said main chain.

These polymers are disclosed, for example, in patent applications EP-A-0 412 704, EP-A-0 412 707, EP-A-0 640 105, WO 95/00578, EP-A-0 582 152 and WO 93/23009 and patents U.S. Pat. No. 4,693,935, U.S. Pat. No. 4,728,571 and U.S. Pat. No. 4,972,037.

These polymers can be amphoteric, anionic or nonionic and they are preferably anionic or nonionic.

Such polymers are, for example, the copolymers capable of being obtained by radical polymerization from the mixture of monomers formed:

a) of 50 to 90% by weight of tert-butyl acrylate,
b) of 0 to 40% by weight of acrylic acid,
c) of 5 to 40% by weight of a silicone macromer of formula: [symbol]
where v is a number ranging from 5 to 700, the percentages by weight being calculated with respect to the total weight of the monomers.

Other examples of grafted silicone polymers are in particular polydimethylsiloxanes (PDMSs) on which are grafted, via a connecting link of thiopropylene type, mixed polymer units of the poly((meth)acrylic acid) type and of the poly(alkyl(meth)acrylate) type and polydimethylsiloxanes (PDMSs) on which are grafted, via a connecting link of thiopropylene type, polymer units of the poly(isobutyl(meth)acrylate) type.

Mention may be made, as other type of silicone fixing polymers, of the product Luviflex® Silk sold by BASF.

The concentration of fixing polymer(s) used in the compositions according to the present invention is between 0.1 and 20% by weight, preferably between 0.5 and 10% by weight, with respect to the total weight of the composition.

### Amps

The polymers in accordance with the invention are represented by polymers comprising at least one ethylenically unsaturated monomer comprising sulfo group in the free or partially or completely neutralized form which can be modified by a fatty chain or copolymers of at least one ethylenically unsaturated monomer comprising sulfo group in the free or partially completely neutralized form and of at least one monomer comprising a hydrophobic part.

The polymers according to the invention can be crosslinked or noncrosslinked.

According to the invention, crosslinked polymers are more particularly preferred.

Preferably, the polymers in accordance with the invention are partially or completely neutralized by an inorganic base (sodium hydroxide, potassium, aqueous ammonia) or an organic base, such as mono-, di- or triethanolamine, an aminomethylpropanediol, N-methylglucamine, basic amino acids, such as arginine and lysine, and the mixtures of these compounds.

### Ethylenically Unsaturated Monomers Comprising a Sulfo Group

They are chosen in particular from vinylsulfonic acid, styrenesulfonic acid, (meth) acrylamido(C₁-C₂₂)alkyl-sulfonic acids, N—(C₁-C₂₂)alkyl((meth)acrylamido(C₁-C₂₂)-alkylsulfonic acids, such as undecylacrylamidomethane-sulfonic acid, and their partially or completely neutralized forms.

Preferably, use is made of (meth)acryl-amido(C₁-C₂₂)alkylsulfonic acids, such as, for example, acrylamidomethanesulfonic acid, acrylamidoethane-sulfonic acid, acrylamidopropanesulfonic acid, 2-acrylamido-2-methylpropanesulfonic acid (AMPS), methacrylamido-2-methylpropanesulfonic acid, 2-acrylamido-n-butanesulfonic acid, 2-acrylamido-2,4,4-trimethylpentanesulfonic acid, 2-methacrylamidododecyl-sulfonic acid, 2-acrylamido-2,6-dimethyl-3-heptane-sulfonic acid, and their partially or completely neutralized forms.

More particularly still, use is made of 2-acrylamido-2-methylpropanesulfonic acid (AMPS) and its partially or completely neutralized forms.

### Monomers Comprising a Hydrophobic Part

When the polymer according to the invention comprises a monomer comprising a hydrophobic part, the hydrophobic part, which is a fatty chain, preferably comprises from 6 to 50 carbon atoms, more preferably from 6 to 22 carbon atoms, more preferably still from 6 to 18 carbon atoms and more particularly from 12 to 18 carbon atoms.

Mention may in particular be made, among the polymers comprising at least one ethylenically unsaturated monomer comprising a sulfo group and not comprising a monomer with a hydrophobic part, of the following homopolymers;
polyacrylamidomethanesulfonic acid,
polyacrylamidoethanesulfonic acid,
polyacrylamidopropanesulfonic acid,
poly(2-acrylamido-2-methylpropanesulfonic acid),
poly(2-methacrylamido-2-methylpropanesulfonic acid),
poly(2-acrylamido-n-butanesulfonic acid), and their partially or completely neutralized forms.

Polymers of this type, and in particular of poly(2-acrylamido-2-methylpropanesulfonic acid), can be crosslinked and partially or completely neutralized. They are generally characterized in that they comprise, distributed randomly:

a) from 90 to 99.9% by weight of units of following formula (I) (AMPS):

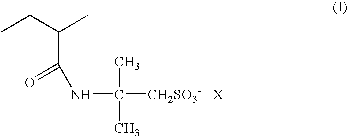

in which X⁺ denotes a proton or an alkali metal cation or an alkaline earth metal equivalent cation or the ammonium cation,
b) from 0.1 to 10% by weight of at least one crosslinking unit having at least two olefinic double bonds,
the proportions by weight being defined with respect to the total weight of the polymer.

Preferably, the crosslinked and neutralized poly(2-acrylamido-2-methylpropanesulfonic acid) comprises from 98 to 99.5% by weight of units of formula (I) and from 0.5 to 2% by weight of crosslinking units.

The crosslinking units having at least two olefinic double bonds are chosen, for example, from dipropylene glycol diallyl ether, polyglycol diallyl ethers, triethylene glycol divinyl ether, hydroquinone diallyl ether, tetraallyloxyethane or other allyl or vinyl ethers of polyfunctional alcohols, tetraethylene glycol diacrylate, triallylamine, trimethylolpropane diallyl ether, methylenebisacrylamide or divinylbenzene.

The crosslinking units having at least two olefinic double bonds are more particularly still chosen from those corresponding to the following general formula (II):

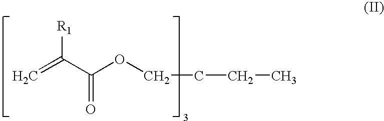

in which R₁ denotes a hydrogen atom or a C₁-C₄ alkyl and more particularly methyl(trimethylolpropane-triacrylate: TMPTA).

The crosslinked and partially or completely neutralized poly(2-acrylamido-2-methylpropanesulfonic acid)s are generally known under the names “Ammonium Polyacrylamido-2-methylpropanesulfonate” or “Ammonium Polyacryloyldimethyltaurate” (INCI dictionary, 2000 edition).

They are, for example, disclosed and prepared in patent application DE-19625810.

They can be obtained according to the known preparation process comprising the following stages:

(a) the ethylenically unsaturated monomer comprising sulfo group in the free form is dispersed or dissolved in a tert-butanol or water and tert-butanol solution;
(b) the monomer solution or dispersion obtained in (a) is neutralized by one or more inorganic or organic bases, preferably aqueous ammonia, in an amount which makes it possible to obtain a degree of neutralization of the sulfonic acid functional groups of the polymer ranging from 0 to 100%;
(c) the crosslinking monomer or monomers is/are added to the solution or dispersion obtained in (b);
(d) a conventional radical polymerization is carried out in the presence of free radical initiators at a temperature ranging from 10 to 150° C.; the polymer precipitating from the solution or dispersion based on tert-butanol.

A particularly preferred product according to the invention is that sold by Clariant under the trade name Hostacerin AMPS; this is a crosslinked poly(2-acrylamido-2-methylpropanesulfonic acid) partially neutralized by aqueous ammonia.

The polymers in accordance with the invention can also be chosen from random AMPS polymers modified by a fatty chain, by reaction with an n-mono(C₆-C₂₂)alkylamine or a di(n-(C₆-C₂₂)alkyl)amine, such as those disclosed in patent application WO 00/31154 (forming an integral part of the content of the description). These polymers can also comprise other hydrophilic ethylenically unsaturated monomers chosen, for example, from (meth)acrylic acids, their β-substituted alkyl derivatives or their esters obtained with monoalcohols or mono- or polyalkylene glycols, (meth)acrylamides, vinylpyrrolidone, maleic anhydride, itaconic acid or maleic acid or the mixtures of these compounds.

Polymers of at least one ethylenically unsaturated monomer comprising sulfo group in the free or partially or completely neutralized form and of at least one monomer with a hydrophobic part.

Such polymers generally have a number-average molecular weight ranging from 1000 to 20 000 000 g/mol, preferably ranging from 20 000 to 5 000 000 g/mol and more preferably still ranging from 100 000 to 1 500 000 g/mol.

When they are crosslinked, the crosslinking agents can be chosen from the compounds possessing olefinic polyunsaturation commonly used for the crosslinking of polymers obtained by radical polymerization.

Mention may be made, for example, of divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl(meth)acrylate, the allyl ethers of alcohols of the sugar series, or other allyl or vinyl ethers of polyfunctional alcohols, and also the allyl esters of phosphoric and/or vinylphosphonic acid derivatives, or the mixtures of these compounds.

Use is made more particularly of methylenebis-acrylamide, allyl methacrylate or trimethylolpropane triacrylate (TMPTA). The degree of crosslinking generally varies from 0.01 to 10 mol % and more particularly from 0.2 to 2 mol %, with respect to the polymer.

Preference is given according to the invention, among said polymers, to the copolymers of AMPS of formula (I) and of at least one hydrophobic monomer having from 6 to 50 carbon atoms and more preferably from 6 to 22 carbon atoms and more preferably still from 6 to 18 carbon atoms and more particularly from 12 to 18 carbon atoms.

These copolymers can additionally comprise one or more nonhydrophobic monomers, such as (meth)acrylic acids, their β-substituted alkyl derivatives or their esters obtained with monoalcohols or mono- or polyalkylene glycols, (meth)acrylamides, vinylpyrrolidone, maleic anhydride, itaconic acid or maleic acid, or the mixtures of these compounds.

Such copolymers are disclosed in particular in patent application EP-A-750 899, in U.S. Pat. No. 5,089,578 and in the following publications by Yotar® Morishima:
“Self-assembling amphiphilic polyelectrolytes and their nanostructures”—Chinese Journal of Polymer Science, Vol. 18, No. 40 (2000), 323-336;
“Micelle formation of random copolymers of sodium 2-(acrylamido)-2-methylpropanesulfonate and a non-ionic surfactant macromonomer in water as studied by fluorescence and dynamic light scattering”, Macromolecules, 2000, Vol. 33, No. 10, 3694-3704;
“Solution properties of micelle networks formed by non-ionic moieties covalently bound to a polyelectrolyte: salt effects on rheological behavior”, Langmuir, 2000, Vol. 16, No. 12, 5324-5332;
“Stimuli responsive amphiphilic copolymers of sodium 2-(acrylamido)-2-methylpropanesulfonate and associative macromonomers”, Polym. Preprint, Div. Polym. Chem., 1999, 40(2), 220-221.

In these specific AMPS copolymers, the monomers comprising a hydrophobic part are preferably chosen from the acrylates or the acrylamides of following formula (III):

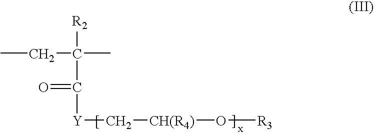

in which R₂ and R₄, which are identical or different, denote a hydrogen atom or a linear or branched C₁-C₆ alkyl radical (preferably methyl); Y denotes O or NII; R₃ denotes a hydrophobic hydrocarbon radical comprising at least from 6 to 50 carbon atoms and more preferably from 6 to 22 carbon atoms and more preferably still from 6 to 18 carbon atoms and more particularly from 12 to 18 carbon atoms; x denotes a number of moles of alkylene oxide and varies from 0 to 100.

The R₃ radical is preferably chosen from C₆-C₁₈ alkyl radicals which are linear (for example, n-hexyl, n-octyl, n-decyl, n-hexadecyl, n-dodecyl), branched or cyclic (for example, cyclododecane (C₁₂) or adamantane (C₁₀)); perfluorinated C₆-C₁₈ alkyl radicals (for example, the group of formula —(CH₂)₂—(CF₂)₉—CF₃); the cholesteryl (C₂₇) radical or a cholesterol ester residue, such as the cholesteryl oxyhexanoate group; or polycyclic aromatic groups, such as naphthalene or pyrene. Preference is more particularly given, among these radicals, to linear alkyl radicals and more particularly to the n-dodecyl radical.

According to a particularly preferred form of the invention, the monomer of formula (III) comprises at least one alkylene oxide unit (x≧1) and preferably a polyoxyalkylene chain. The polyoxyalkylene chain is preferably composed of ethylene oxide units and/or of propylene oxide units and more particularly still is composed of ethylene oxide units. The number of oxyalkylene units generally varies from 3 to 100 and more preferably from 3 to 50 and more preferably still from 7 to 25.

Mention may be made, among these polymers, of:
crosslinked or noncrosslinked and neutralized or unneutralized copolymers comprising from 15 to 60% by weight of AMPS units and from 40 to 85% by weight of (C₈-C₁₆)alkyl(meth)acrylamide units or of (C₈-C₁₆)alkyl (meth)acrylate units with respect to the polymer, such as those disclosed in application EP-A 750 899;
terpolymers comprising from 10 to 90 mol % of acrylamide units, from 0.1 to 10 mol % of AMPS units and from 5 to 80 mol % of n-(C₆-C₁₈)alkylacrylamide units, such as those disclosed in U.S. Pat. No. 5,089,578.

Mention may also be made of the noncrosslinked and crosslinked copolymers of partially or completely neutralized AMPS and of dodecylmethacrylate, and also the noncrosslinked and crosslinked copolymers of partially or completely neutralized AMPS and of n-dodecylmethacrylamide, such as those disclosed in the abovementioned papers by Morishima.

Mention is also more particularly made of the copolymers composed of AMPS units of formula (I) described above and of units of following formula (IV):

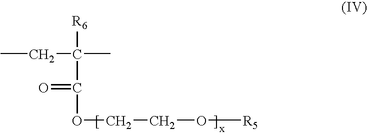

in which x denotes an integer varying from 3 to 100, preferably from 5 to 80 and more preferably from 7 to 25; R₆ has the same meaning as that of R₂ indicated above in the formula (III) and R$ denotes a linear or branched C₆-C₂₂ and more preferably C₁₀-C₂₂ alkyl.

The particularly preferred polymers are those for which x=25, R₆ denotes methyl and R₅ represents n-dodecyl; they are disclosed in the abovementioned papers by Morishima.

The polymers for which X⁺ denotes sodium or ammonium are more particularly preferred.

These preferred polymers can be obtained according to conventional radical polymerization processes in the presence of one or more initiators, such as, for example, azobisisobutyronitrile (AIBN), azobisdimethylvaleronitrile, ABAH (2,2-azobis[2-amidinopropane]hydrochloride), organic peroxides, such as dilauryl peroxide, benzoyl peroxide, tert-butyl hydroperoxide, and the like, inorganic peroxide compounds, such as potassium or ammonium persulfate, or H₂O₂, optionally in the presence of reducing agents.

They are obtained in particular by radical polymerization in a tert-butanol medium, from which they precipitate.

It is possible, by using polymerization by precipitation from tert-butanol, to obtain a distribution in the size of the particles of the polymer which is particularly favorable for its uses.

The distribution and the size of the particles of the polymer can be determined, for example, by laser diffraction or image analysis.

An advantageous distribution for this type of polymer and determined by image analysis is as follows: 60.2% less than 423 microns, 52.0% less than 212 microns, 26.6% less than 106 microns, 2.6% less than 45 microns and 26.6% greater than 850 microns.

The reaction can be carried out at a temperature of between 0 to 150° C., preferably between 10 and 100° C., either at atmospheric pressure or under reduced pressure. It can also be carried out under an inert atmosphere and preferably under nitrogen.

According to this process, 2-acrylamido-2-methylpropanesulfonic acid (AMPS) or either or its sodium and ammonium salts has in particular been polymerized with an ester of (meth)acrylic acid and
of a C₁₀-C₁₈ alcohol oxyethylenated by 8 mol of ethylene oxide (Genapol® C-080 from Hoechst/Clariant),
of a C₁₁ oxo alcohol oxyethylenated by B mol of ethylene oxide (Genapol® UD-080 from Hoechst/Clariant),
of a C₁₁ oxo alcohol oxyethylenated by 7 mol of ethylene oxide (Genapol® UD-070 from Hoechst/Clariant),
of a C₁₂-C₁₄ alcohol oxyethylenated by 7 mol of ethylene oxide (Genapol® LA-070 from Hoechst/Clariant),
of a C₁₂-C₁₄ alcohol oxyethylenated by 9 mol of ethylene oxide (Genapol® LA-090 from Hoechst/Clariant),
of a C₁₂-C₁₄ alcohol oxyethylenated by 11 mol of ethylene oxide (Genapol® LA-110 from Hoechst/Clariant),
of a C₁₆-C₁₈ alcohol oxyethylenated by 8 mol of ethylene oxide (Genapol® T-080 from Hoechst/Clariant),
of a C₁₆-C₁₈ alcohol oxyethylenated by 15 mol of ethylene oxide (Genapol® T-150 from Hoechst/Clariant),
of a C₁₆-C₁₈ alcohol oxyethylenated by 11 mol of ethylene oxide (Genapol® T-150 from Hoechst/Clariant),
of a C₁₆-C₁₈ alcohol oxyethylenated by 20 mol of ethylene oxide (Genapol® T-200 from Hoechst/Clariant),
of a C₁₆-C₁₈ alcohol oxyethylenated by 25 mol of ethylene oxide (Genapol® T-250 from Hoechst/Clariant),
of a C₁₈-C₂₂ alcohol oxyethylenated by 25 mol of ethylene oxide and/or of a C₁₆-C₁₈ iso alcohol oxyethylenated by 25 mol of ethylene oxide.

The molar concentration in % of the units of formula (I) and of the units of formula (IV) in the polymers according to the invention can vary between 0.1 and 99.9 mol %.

Preferably, for the most hydrophobic polymers, the molar proportion of units of formula (III) or (IV) varies from 50.1 to 99.9%, more particularly from 70 to 95% and more particularly still from 80 to 90%.

Preferably, for the polymers which are not very hydrophobic, The molar proportion of units of formula (III) or (IV) varies from 0.1 to 50%, more particularly from 5 to 25% and more particularly still from 10 to 20%.

The distribution of the monomers in the polymers of the invention can be, for example, alternating, block (including multiblock) or random.

According to the invention, it is preferable for the polymers to have heat-sensitive pendant chains and to exhibit an aqueous solution having a viscosity which, beyond a certain threshold temperature, increases or remains virtually constant when the temperature increases.

More particularly still, preference is given to the polymers exhibiting an aqueous solution having a viscosity which is low below a first threshold temperature and which, above this first threshold temperature, increases towards a maximum when the temperature increases and which, above a second threshold temperature, again decreases when the temperature increases. From this viewpoint, it is preferable for the viscosity of the polymer solutions below the first threshold temperature to be from 5 to 50%, in particular from 10 to 30%, of the maximum viscosity at the second threshold temperature.

These polymers preferably result, in water, in a phenomenon of phase separation by heating, reflected by curves exhibiting, as a function of the temperature and of the concentration, a minimum known as the LCST (Lower Critical Solution Temperature).

The viscosities (measured at 25° C. with a Brookfield viscometer, spindle 7) of the 1% aqueous solutions preferably range from 20 000 mPa·s to 100 000 mPa·s and more particularly from 60 000 mPa·s to 70 000 mPa·s.

The AMPS polymers in accordance with the invention can be present in the compositions according to the invention in concentrations ranging from approximately 0.01 to 30% by weight, preferably from 0.1 to 20% by weight and more particularly from 0.5 to 15% by weight, with respect to the total weight of the composition.

### Aerosol

The propellant can be any liquefiable gas generally used in aerosol devices, such as dimethyl ether, C₃₋₅ alkanes, 1,1-difluoroethane, mixtures of dimethyl ether and of C₃₋₅ alkanes, and mixtures of 1,1-difluoroethane and of dimethyl ether and/or of C₃₋₅ alkanes. Preference is very particularly given to the use, as propellant, of dimethyl ether. The propellant is preferably present at more than 20% and preferably between 20 and 70% and more preferably still between 20 and 50% of the total weight of the mixture (propellant (a)+styling composition (b)) present in the aerosol device.

Although the styling compositions present in the aerosol device of the present invention are preferably devoid of lower alcohols, they can comprise up to 50% by weight and preferably up to 25% by weight, with respect to the total weight of the mixture present in the aerosol device, of one or more lower alcohols, such as ethanol or isopropanol.

The styling composition according to the invention can additionally comprise at least one adjuvant chosen from silicones in the soluble, dispersed or microdispersed form, nonionic, anionic, cationic and amphoteric surface-active agents, additional polymers other than the fixing polymers and the AMPS polymers used in the compositions according to the invention, ceramides and pseudoceramides, vitamins and provitamins, including panthenol, vegetable, animal, mineral and synthetic oils, waxes other than ceramides and pseudoceramides, water-soluble and fat-soluble sunscreens which may or may not comprise a silicone portion, solid particles, such as, for example, colored or colorless inorganic and organic pigments, pearlescent and opacifying agents, glitter, active particles, dyes, sequestering agents, plasticizing agents, solubilizing agents, acidifying agents, basifying agents, neutralizing agents, inorganic and organic thickening agents, antioxidants, hydroxy acids, penetrating agents, fragrances and preservatives.

A person skilled in the art will take care to choose the optional additives and their amounts so that they do not interfere with the properties of the compositions according to the present invention.

These additives are present in the composition according to the invention in an amount ranging from 0 to 20% by weight, with respect to the total weight of the composition.

In the styling compositions present in the aerosol device, the water represents from 20 to 80% by weight, with respect to the total weight of the mixture present in the aerosol device.

Of course, a person skilled in the art will take care to choose this or these optional additional additives and/or their amounts so that the advantageous properties intrinsic to the invention are not detrimentally affected by the envisaged addition or additions.

With this specific combination, the hairstyle is shaped by working with the fingers without obtaining the instantaneous hardening and the instantaneous setting conventionally encountered with fixing products. Furthermore, it is possible to obtain, on the fingers and on the hand, a lasting crackling effect which confers a novel rheological nature on the composition, contributing a feeling of freshness on application. Specifically, this composition exhibits the advantage of spreading out well over the hair. Application can be carried out both on wet hair and on dry hair and directly or via the fingers. For application to wet hair, drying in the open air or with heat can be carried out.

The styling result is supple and natural.

The formulation example illustrates the present invention without, however, limiting it.

### EXAMPLE

Aerosol comprising, as g/100 g:
PVP/VA (BASF): 3.25
Polyacrylamidomethylpropanesulfonic acid partially neutralized with aqueous ammonia and highly crosslinked, provided under the name Aristoflex
HMS by Clariant: 0.97
Water: 39.65
Alcohol: 21.13
DME: 35.00

This composition “crackles” in the hand and induces an effect of freshness. It makes possible, after drying, lasting form retention of the hairstyle.

What is claimed is:

1. A composition packaged in an aerosol device, comprising
(a) at least one propellant and
(b) a styling composition comprising at least (i) one nonpolyurethane anionic or nonionic fixing polymer and (ii) at least one polymer comprising at least one ethylenically unsaturated monomer comprising a sulfo group in the free or partially or completely neutralized form.

2. The composition as claimed in claim 1, characterized in that the ethylenically unsaturated monomer comprising a sulfo group is chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids, and their partially or completely neutralized forms.

3. The composition as claimed in claim 2, characterized in that the ethylenically unsaturated monomer comprising a sulfo group is chosen from acrylamidomethanesulfonic acid, acrylamidoethane-sulfonic acid, acrylamidopropanesulfonic acid, 2-acrylamido-2-methylpropanesulfonic acid (AMPS), methacrylamido-2-methylpropanesulfonic acid, 2-acrylamido-n-butanesulfonic acid, 2-acrylamido-2,4,4-trimethylpentanesulfonic acid, 2-methacrylamidododecyl-sulfonic acid, 2-acrylamido-2,6-dimethyl-3-heptane-sulfonic acid, and their partially or completely neutralized forms.

4. The composition as claimed in claim 3, characterized in that the ethylenically unsaturated monomer comprising a sulfo group is 2-acrylamido-2-methylpropanesulfonic acid (AMPS) and its partially or completely neutralized forms, of following formula (I): 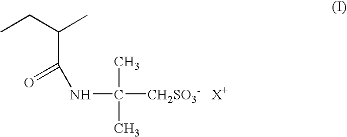
in which X⁺ is a proton or an alkali metal cation or an alkaline earth metal cation or the ammonium ion.

5. The composition as claimed in any one of the preceding claims, characterized in that the polymer additionally comprises at least one hydrophobic part.

6. The composition as claimed in claim 5, characterized in that the hydrophobic part of the polymer comprises from 6 to 50 carbon atoms.

7. The composition as claimed in claim 6, characterized in that the hydrophobic part of the polymer comprises from 6 to 22 carbon atoms.

8. The composition as claimed in any one of the preceding claims, characterized in that the polymers are partially or completely neutralized by an inorganic or organic base.

9. The composition as claimed in any one of the preceding claims, characterized in that the polymers are crosslinked or noncrosslinked.

10. The composition as claimed in claim 9, characterized in that the polymers are crosslinked.

11. The composition as claimed in any one of claims 1 to 4 and 10, characterized in that the polymer is crosslinked and partially or completely neutralized poly(2-acrylamido-2-methylpropanesulfonic acid).

12. The composition as claimed in any one of claims 11, characterized in that the polymer is chosen from copolymers of 2-acrylamido-2-methylpropanesulfonic acid (AMPS) of formula (I) and of at least one hydrophobic monomer.

13. The composition as claimed in claim 12, characterized in that the hydrophobic monomer is chosen from the acrylates or the acrylamides of following formula (III): 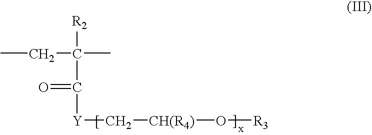
in which R₂ and R₄, which are identical or different, denote a hydrogen atom or a linear or branched C₁-C₈ alkyl radical (preferably methyl); Y denotes O or NH; R₃ denotes a hydrophobic hydrocarbon radical comprising at least from 6 to 50 carbon atoms, preferably from 6 to 22 carbon atoms, more preferably still from 6 to 18 carbon atoms and more particularly from 12 to 18 carbon atoms; x denotes a number of moles of alkylene oxide and varies from 0 to 100.

14. The composition as claimed in claim 13, characterized in that the hydrophobic radical R₃ is chosen from C₆-C₁₈ alkyl radicals which are linear, branched or cyclic; perfluorinated C₆-C₁₈ alkyl radicals; the cholesteryl radical or a cholesterol ester; or polycyclic aromatic groups.

15. The composition as claimed in any one of claims 12 to 14, characterized in that the polymer is chosen from copolymers of 2-acrylamido-2-methylpropanesulfonic acid (AMPS) of formula (I) and of at least one hydrophobic monomer of following formula (IV): 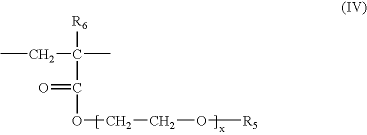
in which x denotes an integer varying from 3 to 100, preferably from 5 to 80 and more preferably from 7 to 25; R₆ has the same meaning as that of R₂ indicated above in the formula (III) and R₅ denotes a linear or branched C₆-C₂₂ and more preferably C₁₀-C₂₂ alkyl.

16. The composition as claimed in any one of claims 12 to 16, characterized in that the polymer is a neutralized copolymer of 2-acrylamido-2-methylpropanesulfonic acid (AMPS) of formula (I) and of n-dodecylacrylamide [formula (III), where x=0, R₂═H, Y═NH, R₃=n-C₁₂].

17. The composition as claimed in any one of claims 12 to 16, characterized in that the polymer is a crosslinked copolymer of 2-acrylamido-2-methylpropanesulfonic acid (AMPS) of formula (I) neutralized by NH₃ and of units of formula (IV) in which R₆ denotes hydrogen or a methyl radical, R₅═C₁₆-C₁₈ and x−b 25.

18. The composition as claimed in any one of the preceding claims, characterized in that the polymer (ii) represents from 0.01 to 30% by weight, preferably from 0.1 to 20% by weight and more particularly from 0.5 to 15% by weight, of the total weight of the composition.

19. The composition as claimed in any one of the preceding claims, characterized in that the anionic fixing polymer is chosen from homopolymers or copolymers of acrylic or methacrylic acid or their salts, copolymers of crotonic acid, copolymers of C₄-C₈ monounsaturated carboxylic acids or anhydrides, polyacrylamides comprising carboxylate groups, homopolymers or copolymers comprising sulfo groups, and anionic grafted silicone polymers.

20. The composition as claimed in any one of the preceding claims, characterized in that the nonionic fixing polymer is chosen from polyalkyloxazolines, vinyl acetate homopolymers and copolymers, acrylic ester homopolymers and copolymers, acrylonitrile copolymers, styrene homopolymers and copolymers, polyamides, vinyllactam homopolymers other than vinylpyrrolidone homopolymers, vinyllactam copolymers and nonionic grafted silicone polymers.

21. The cosmetic composition as claimed in one of the preceding claims, such that the concentration of fixing polymer(s) (i) is between 0.1 and 20% by weight, preferably between 0.5 and 10% by weight, with respect to the total weight of the composition.

22. The composition as claimed in any one of the preceding claims, characterized in that the water represents from 20 to 80% by weight, with respect to the total weight of the mixture present in the aerosol device.

23. The composition as claimed in any one of the preceding claims, characterized in that the propellant (a) represents from 20% to 70% of the total weight of the mixture present in the aerosol device and preferably between 20 and 50%.

24. The composition as claimed in any one of the preceding claims, characterized in that the propellant is chosen from C₃-C₅ alkanes, difluoroethane, dimethyl ether or their mixtures.

25. The composition as claimed in claim 24, characterized in that the propellant is dimethyl ether.

26. The composition as claimed in any one of the preceding claims, characterized in that it is a hair lacquer.

27. The composition as claimed in any one of the preceding claims, characterized in that it additionally comprises at least one adjuvant chosen from silicones in the soluble, dispersed or microdispersed form, nonionic, anionic, cationic and amphoteric surface-active agents, additional polymers other than the fixing polymers or the polyAMPs used in the compositions according to the invention, ceramides and pseudoceramides, vitamins and provitamins, including panthenol, vegetable, animal, mineral and synthetic oils, waxes other than ceramides and pseudoceramides, water-soluble and fat-soluble sunscreens which may or may not comprise a silicone portion, solid particles, such as, for example, colored or colorless inorganic and organic pigments, pearlescent and opacifying agents, glitter, active particles, dyes, sequestering agents, plasticizing agents, solubilizing agents, acidifying agents, basifying agents, neutralizing agents, inorganic and organic thickening agents, antioxidants, hydroxy acids, penetrating agents, fragrances and preservatives.

28. A method for styling the hair, which consists in vaporizing the mixture present in the aerosol device as claimed in any one of the preceding claims directly over the hair to be shaped or via the fingers and in allowing the hair thus treated to dry.
